(19) United States
(12) Patent Application Publication
(10) Pub. No.: US 2004/0181049 A1
(43) Pub. Date: Sep. 16, 2004

(54) Rna purification methods

(76) Inventors: Michael S. Orr, Gaithersburg, MD (US); Daniel Bednarik, Gaithersburg, MD (US); Daniel J Wilson, Gaithersburg, MD (US)

Correspondence Address: MORGAN LEWIS & BOCKIUS LLP, 1111 PENNSYLVANIA AVENUE NW, WASHINGTON, DC, 20004, US

(21) Appl. No.: 10/479,724

(22) PCT Filed: Jun. 5, 2002
(86) PCT No.: PCT/US02/17615

Related U.S. Application Data
(60) Provisional application No. 60/295,575, filed on Jun. 5, 2001.

(51) Int. Cl. C07H 21/02

(52) U.S. Cl. 536/25.4

(57) ABSTRACT

Methods for the preparation and isolation of high quality RNA are described. The methods do not require lysis of red blood cells and may be used to isolate high quality RNA from cells such as epithelial cells, fibroblasts, bone marrow progenitors, brain tissue or brain cells. The present invention is also directed to kits for practicing the disclosed high quality RNA isolation methods.

### RELATED APPLICATIONS

The present application claims priority to U.S. Provisional Application 60/295,575, filed Jun. 5, 2001, which is herein incorporated by reference its entirety.

### FIELD OF THE INVENTION

The instant invention includes methods of isolating high quality RNA from cells such as epithelial cells, fibroblasts, bone marrow progenitors or brain tissue. Also included are kits containing reagents and other materials useful for practicing the RNA isolation methods of the invention.

### BACKGROUND OF THE INVENTION

Isolation of intact, high quality RNA is useful for many molecular biological procedures. For example, high quality RNA can be used in gene expression analyses in microarrays, cDNA synthesis, in vitro translation or Northern hybridization. See, for example, J. Sambrook et al., Molecular Cloning: A Laboratory Manual (Cold Spring Harbor 1989). High quality RNA can also be used in clinical practice to diagnose infections, detect the presence of cells expressing oncogenes or any other gene of interest, detect familial disorders, and monitor the state of host defense mechanisms. The critical factor in all of these methods is the ability to obtain high quality RNA.

Current methods for isolating RNA include a variety of techniques to disrupt the cell and liberate RNA into solution and to protect RNA from RNases. Following these steps, the RNA is separated from the DNA and protein which is solubilized along with the RNA. The use of the powerfully chaotropic salts of guanidinium to simultaneously lyse cells, solubilize RNA and inhibit RNases was described in Chirgwin et al., Biochem., 18:5294-5299 (1979). Other methods free solubilized RNA of contaminating protein and DNA by extraction with phenol at an acidic pH using chloroform to effect a phase separation (D. M. Wallace (1987), Meth. Enzym., 15:33-41).

A commonly used single step isolation of RNA involves homogenizing cells in 4M guanidinium isothiocyanate, followed by the sequential addition of sodium acetate (pH 4) phenol, and chloroform/isoamyl alcohol. After centrifugation, RNA is precipitated from the upper layer by the addition of alcohol (P. Chomczynski and N. Sacchi, (1987) Anal. Biochem., 162:156-159-and “Preparation and Analysis of RNA” (1991) in Current Protocols in Molecular Biology, Unit 4.2 (Supplement 14), ed. F. M. Ausubel et al., John Wiley). Less commonly used methods include the addition of hot phenol to a cell suspension, followed by alcohol precipitation (T. Maniatis et al., (1982) Molecular Cloning, A Laboratory Manual, Cold Spring Harbor Laboratory); the use of anionic or non-ionic surfactants to lyse cells and liberate cytoplasmic RNA; and the use of inhibitors of RNases such as vanadyl riboside complexes and diethylpyrocarbonate (L. G. Davis et al., “Guanidine Isothiocyanate Preparation of Total RNA” and “RNA Preparation: Mini Method” (1991) in Basic Methods in Molecular Biology, Elsevier, N.Y., pp. 130-138). U.S. Pat. No. 4,843,155 to Chomczynski, describes a method in which a stable mixture of phenol and guanidinium salt at an acidic pH is added to the cells. After phase separation with chloroform, the RNA in the aqueous phase is recovered by precipitation with an alcohol.

RNA purification protocols is are commercially available for isolating RNA from bone marrow progenitors (Qiagen RNeasy Midi Column). Importantly, this procedure requires pre-treatment and lysis of red blood cells (RBC) in a mixed population of bone marrow progenitors before RNA isolation. Pre-treatment and lysis of red blood cells is a major disadvantage to this procedure because reports have indicated that RBC, membrane components, or heam complexes from erythrocytes can act as mitogenic or co-mitogenic agents for leukocytes (Johnson et al. (1972) Augmentation of phytohemagglutinin mitogenic activity by erythrocyte membranes. Cell. Immunol. 3(2) 186-97; Kalechman et al. (1993) Enhancing effects of autologous erythrocytes on human or mouse cytokine secretion and IL-2R expression. Cell. Immunol. 148(1):114-29; Stenzel et al. (1981) Mitogenic and co-mitogenic properties of hemin. J. Immunol. 127(6):2469-73; Tarnvik (1970) A role for red cells in phytohaemagglutinin-induced lymphocyte stimulation. Acta Pathol. Microbiol. Scand [B] Microbiol. Immunol. 78(6):733-40). The mitogenic activity of the pre-treated, lysed red blood cells is a disadvantage in RNA purification from cell samples because it can cause the gene expression profile of the cell sample whose RNA is being extracted to be distorted by the RNA purification procedure itself.

There remains a need in the art for a simple method for isolating high quality RNA from cells such as epithelial cells, fibroblasts, bone marrow progenitors or brain cells, which minimizes hydrolysis and degradation of the RNA and also minimizes alteration of gene expression caused by pre-treatment and lysis of the RBCs in the sample. The isolated high quality RNA can be used for clinical, diagnostic or experimental purposes.

### SUMMARY OF THE INVENTION

The present invention includes methods for isolating high quality RNA from cell or tissue samples, including epithelial cells, fibroblasts, bone marrow progenitors or brain tissue without pre-treatment and lysis of red blood cells present in the mixture of cells before RNA isolation.

The ability to obtain high quality RNA is essential for microarray experiments. Previous attempts using commercially available RNA isolation techniques failed to isolate intact RNA from bone marrow progenitors because the RNA was degraded. The methods of the invention are amenable to isolating RNA in a production environment because they are relatively simple and quick and the isolated RNA is much less degraded than RNA prepared using available RNA purification techniques.

In one embodiment, the present invention includes a method of isolating high quality RNA from a cell or tissue sample comprising the steps of: a) suspending the cell or tissue sample in a buffer without pre-lysing red blood cells in the sample, b) lysing the cell or tissue sample and c) isolating high quality RNA from the lysed sample.

In another embodiment, the present invention includes a method for isolating high quality RNA from a cell or tissue sample comprising the steps of: a) suspending the cell or tissue sample in a buffer without pre-lysing red blood cells in the sample, b) suspending cells in a solution of a chaotropic agent; c) adding a cell lysis reagent and d) isolating high quality RNA.

Methods of the invention may employ cell suspension techniques including but not limited to using a pipette or a syringe attached to a needle and repeatedly syringing or pipetting the sample.

Another embodiment of the invention includes using chaotropic agents such as urea, guanidine thiocyanate or guanidine isothiocyanate having a concentration of approximately 4 Molar. In an embodiment, the chaotropic agent contains approximately 1% 2-mercaptoethanol.

Another embodiment of the invention includes using cell lysis reagents such as TRIZOL.

The RNA purification methods of the invention may be used to isolate high quality RNA from many different cell types including but not limited to epithelial cells, fibroblasts, bone marrow cells, bone marrow progenitors, brain cells or brain tissue including human types of all of these cells.

Another embodiment of the invention includes a method for isolating high quality RNA from bone marrow cells comprising the steps of: a) suspending bone marrow cells in a buffer without lysing red blood cells in the sample, b) combining bone marrow cells with an approximately 4 Molar solution of guanidine thiocyanate containing approximately 1% 2-mercaptoethanol, c) suspending said bone marrow cells in said solution of step b) using a pipette or a syringe attached to a needle, d) adding TRIZOL reagent and e) isolating high quality RNA.

Another embodiment of the invention includes a method for isolating high quality RNA from bone marrow cells comprising the steps of: a) suspending cells in a buffer by repeated pipetting or repeated syringing without lysing red blood cells present in the sample, b) centrifuging the solution of step a) to form a pellet, c) resuspending said pellet in a buffer by repeated pipetting or repeated syringing, d) adding TRIZOL reagent and, e) isolating high quality RNA.

Another embodiment of the invention includes a method for isolating high quality RNA from brain cells or brain tissue comprising the steps of: a) suspending brain cells or brain tissue in a buffer without lysing red blood cells in the sample, b) suspending said brain cells or brain tissue in a solution of about 6M guanidine isothiocyanate containing about 4% beta-mercaptoethanol by repeated syringing or pipetting, c) adding TRIZOL to the sample and d) isolating high quality RNA.

Another embodiment of the present invention includes a kit for isolating high quality RNA from a cell sample comprising a) a vial or container of a solution of a chaotropic agent, which in certain embodiments is a solution of about 4 Molar or about 6 Molar guanidine thiocyanate, and wherein said solution of a chaotropic agent also contains about 1% or about 4% by volume 2-mercaptoethanol, b) a vial or container of a solution of a cell lysis reagent, c) optionally syringes and syringe needles, and d) a package insert with instructions for isolating high quality RNA from a cell sample.

Another embodiment of the present invention includes a method for predicting the bone marrow toxicity of a substance using gene expression comprising: a) obtaining a cell sample that has been previously treated with a test agent or isolated from an organism treated with a test agent, b) isolating high quality RNA from the sample without pre-lysing red blood cells present in the sample prior to lysing the rest of the cells in the sample, c) isolating high quality RNA from a cell or tissue sample that has not been exposed to the test agent, without pre-lysing red blood cells present in the sample, and d) correlating differences in the gene expression profiles between the two samples with the toxicity of the substance.

Another embodiment of the present invention includes a method for predicting the bone marrow toxicity of a substance using gene expression profiling as described above, wherein said cell sample is bone marrow progenitors.

### BRIEF DESCRIPTION OF THE FIGURES

FIG. 1 shows an Agilent 2100 Bioanalyzer comparison of rat femur RNA samples isolated using the methods and procedures of the present invention compared to rat femur RNA isolated using the Qiagen Rneasy procedure. Sample 3 is high quality rat femur RNA isolated using Protocol Number Two of the invention (rRNA Ratio [28S/18S] 2.51); Sample 5 is rat femur RNA isolated using the Qiagen Rneasy Protocol without RBC lysis (rRNA Ratio [28S/18S]=0.00); Sample 6 is rat femur RNA isolated using the Qiagen Rneasy Protocol with RBC lysis (rRNA Ratio [28S/18S]=0.00); Sample 7 is high quality rat femur RNA isolated using Protocol Number One of the invention (rRNA Ratio [28S/18S]=2.32); Sample 12 is high quality rat hepatocyte control RNA (rRNA Ratio [28S/18S]=2.02).

FIG. 2 shows an RNA separation gel containing 2.5% agarose. Sample 5 and Sample 6 correspond to the rat femur RNA samples isolated using the Qiagen Rneasy procedure and depicted as Sample 5 and Sample 6 in FIG. 1. The prominent bands in each of Sample 5 and Sample 6 are contaminating genomic DNA in each of the samples.

### DETAILED DESCRIPTION

General Description

The methods for isolating high quality RNA of the instant invention do not require pre-treatment and lysis of red blood cells before RNA isolation. The present invention also has the advantage of not requiring multiple steps for RNA isolation, saving time and decreasing the probability that gene expression modulations are produced from the RNA isolation procedure itself. The present invention has the added advantage that it also captures all of the different cell types present in a sample, allowing a more global analysis of gene expression modulation in a mixed population of cells.

An additional aspect of the present invention is that since red blood cells are not lysed prior to RNA isolation, the presence of differentiating erythrocytes enhances the ability to predict bone marrow toxicity differences in various samples.

The present invention includes methods for isolating high quality RNA from cells such as epithelial cells, fibroblasts, bone marrow progenitors or brain tissue. The present methods may also be practiced using any other tissue or cell type found in animals.

In one embodiment, the present methods included the isolation of high-quality RNA derived from bone marrow progenitors obtained from femur. The bone marrow progenitors may be isolated, for example, from rat femur, or femur from any other animal such as dog, cat, goat, cow, sheep, primate, rodent, human, pig, horse or any other mammal. The bone marrow may be isolated from any other bone in the body of an animal that produces bone marrow. Similarly, the methods for isolating high quality RNA of the present invention may be applied to any cell type isolated from any organism that produces RNA.

In another embodiment, the present methods include the isolation of high-quality RNA from brain cells or brain tissue. The brain cells or brain tissue may be isolated from, for example, rat or any other animal such as dog, cat, goat, cow, sheep, primate, rodent, human, pig, horse or any other mammal.

In another embodiment, the methods for isolating high quality RNA of the present invention are based on the finding that standard RNA isolation procedures from eukaryotic cells may be dramatically improved by suspending the cells prior to RNA extraction in a solution of a chaotropic agent such as solutions of guanidine thiocyanate containing 2-mercaptoethanol. Suspension of the cells is accomplished by repeated suspension, at least about five times or more, using a syringe attached to a syringe needle or a pipette. Following this suspension, the cell lysate is mixed with a solution of a cell lysis reagent, such as TRIZOL reagent, after which any RNA extraction procedure may be used to complete the isolation of high quality RNA. The methods of the invention do not require pretreatment of the cell sample to lyse RBCs.

In another embodiment, the cells from which the RNA is to be extracted may be suspended in a buffer, such as RPMI, by suspension at least about 5 times or more using a syringe attached to a syringe needle or a pipette. The cells are then pelleted and resuspended in a buffer, such as RPMI, and lysed using a cell lysis reagent, such as TRIZOL reagent. After this, any RNA extraction procedure may be us to complete the isolation of high quality RNA. The methods of the invention do not require pretreatment of the cell sample to lyse RBCs.

The invention also includes kits containing reagents in appropriate volumes and materials such as syringes, needles, and pipettes useful for practicing the methods of the invention for the isolation of high quality RNA from eukaryotic or prokaryotic organisms.

Definitions

It must be noted that as used in this specification and the appended claims, the singular forms “a”, “an” and “the” include plural referents unless the context clearly dictates otherwise. Thus, for example, reference to “a method for isolating high quality RNA” includes one or more methods or steps of the type described herein, reference to “a kit for isolating high quality RNA” includes large numbers of kits for isolating high quality RNA.

As used herein, a “chaotropic agent” includes any chemical, such as urea or guanidine hydrochloride, that can disrupt the hydrogen bonding structure of water. In concentrated solutions they can denature proteins because they reduce the hydrophobic effect. Chaotropic agents include guanidine thiocyanate and guanidine isothiocyanate. Concentrated solutions of chaotropic agents are greater than about 1 Molar, or greater than about 2 Molar, or greater than about 3 Molar, or greater than about 4 Molar, or greater than about 5 Molar, or greater than about 6 Molar (greater than about 1 Molar to about 6 Molar).

As used herein, a “cell lysis reagent” is any chemical or combination of chemicals that causes cell lysis to occur when brought in contact with a sample of cells. Cell lysis reagents include solutions of sodium hydroxide with a detergent such as sodium dodecylsulfate (SDS) or CHAPS, TRIZOL, formulations of lytic enzymes, such as lysozyme, as well as many other cell lysis reagents that are known in the field of RNA purification.

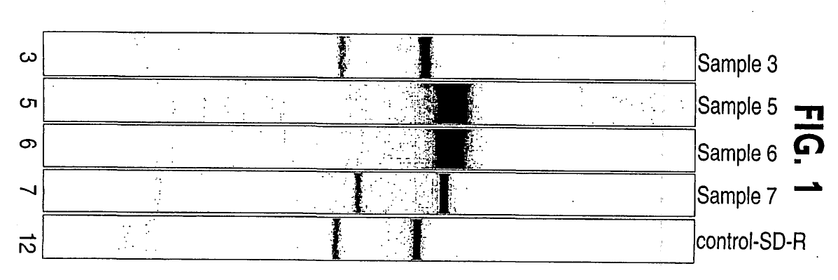

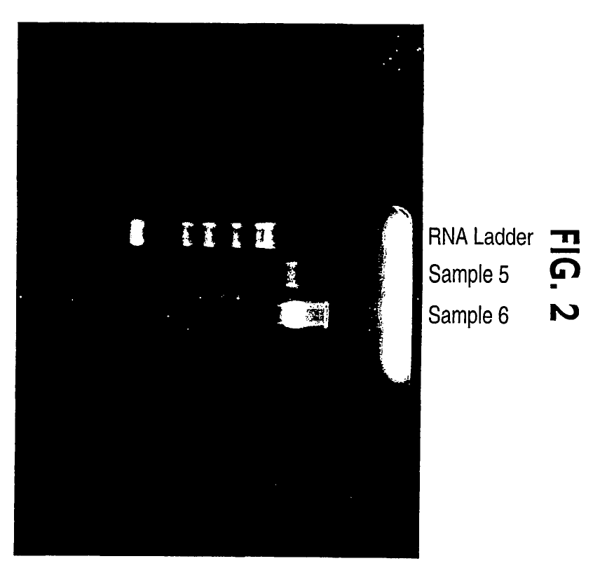

As used herein, the term “high quality RNA” refers to isolated and purified RNA that has fluorescence intensity or absorbance ratio of 28S/18S RNA of greater than about 1.50, or greater than about 1.55, or greater than about 1.60, or greater than about 1.65, or greater than about 1.70 or greater than about 1.75, or greater than about 1.80, or greater than about 1.85, or greater than about 1.90, or greater than about 1.95, or greater than about 2.00 (greater than about 1.5 to about 2.0); wherein said high quality RNA is not significantly degraded by Rnase or significantly contaminated with DNA. High quality total eukaryotic rRNA has intact 28s and 18s ribosomal bands. The ratio of the 28s to 18s for good quality RNA is usually greater than about 1.5. The higher the ratio of 28S to 18S RNA the better the quality of the RNA (better quality RNA is intact RNA lacking degradation by RNases). Extremely high quality rRNA has a ratio of 28s/28s of greater than 2.0. The lack of intact bands indicates considerable quantities of degradation (see samples 5 and 6 in FIG. 1). Furthermore, large bands (the ones closest to the wells) are DNA contamination present in the isolated RNA (see FIG. 2).

As used herein, “RPMI” refers to RPMI (Roswell Park Memorial Institute) Media which are enriched formulations with extensive applications for mammalian cells. These media were originally formulated for suspension cultures or monolayer cultures of human leukemia cells. RPMI media are available from Invitrogen Life Technologies.

As used herein, the term “significantly contaminated with genomic DNA” refers to a purified RNA sample that contains greater than about 5% or about 10% or about 15% or about 20% or about 25% or about 30% or about 35% or about 40% or about 45% or about 50% DNA (about 5% to about 50% or more DNA).

As used herein, the term “significantly degraded by RNAse” means an RNA sample that has been degraded by Rnase digestion such that it has been digested by greater than about 5% or about 10% or about 15% or about 20% or about 25% or about 30% or about 35% or about 40% or about 45% or about 50% (about 5% to about 50% or more) compared to an undigested RNA sample.

As used herein “TRIZOL” or “TRIZOL reagent” is a mono-phasic solution of phenol and guanidine isothiocyanate. TRIZOL is sold by Invitrogen Life Technologies and is described in U.S. Pat. No. 5,346,994.

RNA Purification Methods

Once a cell lysate has been produced using the methods of the present invention, any method for purifying RNA from a cell lysate may be employed. The methods for producing high quality RNA of the present invention do not require a pre-lysis step or a pre-treatment of the cell or tissue sample to lyse red blood cells prior to lysing the rest of the cells in the sample.

Commonly employed RNA purification systems include those commercially available from such vendors as Qiagen, Promega, Stratagene, Roche Applied Science and others.

Kits

The invention includes any or all of the reagents, syringe needles, syringes, pipettes and tubes useful for practicing the disclosed high quality RNA purification methods packaged together in a kit or container. Formulations or compositions of the invention may be packaged together with, or included in a kit with instructions or a package insert referring to a high quality RNA purification method of the invention. For instance, such instructions or package inserts may address recommended storage conditions, such as time, temperature and exposure to light for use in a laboratory or clinical setting. Such instructions or package inserts may also address the particular advantages of the RNA purification methods of the invention, such as the ease of storage for formulations that may require use in the field, outside of controlled hospital, clinic, laboratory or office conditions.

Without further description, it is believed that a person of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### EXAMPLES

### Example I

### Protocol Number One for Isolation of High Quality RNA From Bone Marrow

Protocol Number One consists of lysing bone marrow progenitors (without pre-lysing red blood cells) in about 1 mL of about 4M guanidine thiocyanate solution containing about 1% 2-mercaptoethanol. The mixture of cells and lysis buffer is syringed about five times with a 20 Gauge needle attached to a syringe to obtain a thorough suspension of lysate. The lysate of cells and lysis solution are then mixed with about 11 mL of TRIZOL reagent. Any RNA isolation maybe used after these steps.

For Protocol Number One, instead of syringing the cell lysate with a 20-Guage needle, the mixture of cells and lysis buffer may be mixed using a 1 mL pipette by pulling the lysate into the pipette and expelling the mixture about five times. The second alteration is that the mixture of about 4 M guanidine thiocyanate and about 1% 2-mercaptoethanol (1 mL) and the about 11 mL of TRIZOL reagent can be mixed using a 10 mL pipette and pipetting five times.

The lysate mixture of bone marrow progenitors consisting of about 1 mL of the about 4M solution, TRIZOL and lysed cells following syringing procedure may be stored at −80° C. The RNA is stable in this solution and the RNA can be isolated at a later time. High quality RNA may be isolated from cell lysates that have been stored at −80° C. for at least about six months.

### Example II

### Protocol Number Two for Isolation of High Quality RNA From Bone Marrow

Protocol Number Two consists of syringing bone marrow progenitors in RPMI (without pre-lysing red blood cells) with a 20 Gauge needle attached to a syringe about five times before the cells are pelleted. The cells are then pelleted by centrifugation for 10 min at 2000 rpm at 4° C. The pellet has about 1 mL of RPMI medium added to it and the cells are resuspended in the about 1 mL of RPMI by syringing with a 20 Guage needle and syringing about five times to produce a single cell suspension. The cells are immediately lysed in TRIZOL reagent and the RNA is then isolated using any standard RNA isolation procedure.

### Example III

### Production Protocol for Isolation of High Quality RNA From Bone Marrow Cells (4 Molar Guanidine Thiocyanate+TRIZOL)

Bone marrow cells are suspended in RPMI solution (without pre-lysing red blood cells) and kept on ice in 15 mL tubes. The cells are pelleted by centrifuging at 2000 rpm at 4° C. for 10 minutes. Once the cells have been pelleted, the RPMI medium is removed and about 1 mL of 4 M guanidine thiocyanate containing about 1% β-mercaptoethanol solution is added to the pellet of bone marrow cells. The cell lysate is repeatedly syringed at least about 5 times using a 20G syringe. About 1 mL of the cell lysate is added to about 11 L of TRIZOL in a 15 mL tube. The mixture is syringed about 5 times using a 20G needle attached to a 20 cc syringe to homogenize. The tube is then placed on ice. The RNA is isolated using standard RNA isolation procedures.

### Example IV

### Production Protocol for Isolation of High Quality RNA From Rat Bone Marrow Progenitor Cells

Bone marrow cells in RPMI are kept on ice in 15 ml tubes. 500 μl of 2-mercaptoethanol is added to 50 mL of 4 M Guanidine. The tube is mixed by inverting. Cells are spun at 2000 rpm at 4° C. for 10 minutes to pellet. Fresh 50 ml tubes are prepared by adding 11 ml of TRIZOL to each tube. After pelleting cells, RPMI is removed. 1 ml of a 4 M Guanidine Thiocyanate—2-mercaptoethanol solution is added to the pellet of bone marrow cells. The cells are triturated through the syringe at least 5 times to create a single cell suspension. Using the syringe, the cell suspensions are transferred to the appropriate 50 ml conical tube containing TRIZOL. The cells are homogenized in the TRIZOL by triturating the mixture at least 5 times with the syringe. The tubes are placed on ice. The steps beginning from cell pelleting are repeated for all samples. At this point, the samples may be stored in a −80° C. freezer, until full extraction is scheduled.

Full Extraction of RNA From homogenates

The volumes of the different reagents to be used for each sample are calculated using the following ratios: Chloroform: 0.4-ml chloroform per 1 ml of TRIZOL/Guanidine Mix. For 12 ml, the calculation would be 0.4×12=4.8; Isopropanol: 0.5 ml of 100% Isopropanol per 1 ml of TRIZOL/Guanidine Mix. For 12 ml, the calculation would be 0.5 ml×12=6; 75% Ethanol: 1 ml of 75% Ethanol, per 1 ml of TRIZOL/Guanidine Mix. For 12 ml, the calculation would be 1×12=12.

Empty PLG tubes are centrifuged at 1500×g (2000 rpm) @ 4° C. Homogenate is transferred to pre-spun PLG tube. The appropriate volume of Chloroform is added to each sample. Tubes are capped tightly and shaken vigorously for about 15 seconds. The samples are incubated for 10 minutes on ice or in the centrifuge at 4° C. The samples are then centrifuged at 4300 RPM for 7 minutes @ 4° C. The top aqueous phase is transferred (˜60% TRIZOL volume) by pouring into a clean 50 ml conical tube and the appropriate volume of Isopropanol is added. The tubes are capped and mixed by gentle inversion and incubated on ice or in a centrifuge for 30 minutes. The tubes are then centrifuged at 6,760 rpm for 30 minutes at 4° C. The supernatant is then removed. The appropriate volume (0.5 to 1.5 ml) of pre-chilled 75% EtOH is added to the sample tubes. The samples are then spun at 6,980 rpm for 5 minutes at 4° C. Then, leaving approximately 1 ml of 75% EtOH in the tube, the 75% EtOH supernatant is poured off. The remaining 75% ETOH and pellet are transferred to an appropriately labeled 1.5 ml microcentrifuge tube. The samples are spun in an Eppendorf centrifuge at 14,000 RPM for 5 min at 4° C. The 75% EtOH is removed and the samples are pulse spun in a picofuge. The residual 75% EtOH is removed and the pellets are air-dried at room temperature for 10 minutes. While the pellets are drying, a sufficient volume of RNAsecure is heated at approximately 65° C. for at least 10 min. After drying, each pellet is resuspended in an appropriate volume of RNAsecure, which is typically in the range of 12 μl to 200 μl. All samples are incubated at approximately 65° C. for at least 10 minutes, in a heat block. The samples are stored in a −152° C. freezer.

### Example V

### Isolation of High Quality RNA From Human Brain

Guanidine Isothiocyanate is prepared for use (GITC+BME) by adding 0.04 volumes of 2-mercaptoethanol to 1 volume* of 6 M Guanidine Thiocynate. (*The volume amount depends upon the number of samples to be prepped (approximately 525 μl per sample). The tube is thoroughly mixed by inverting several times. The solution is allowed to sit at room temperature for 30 minutes to allow reduction of the entire GITC solution.

Each sample is processed individually in the following manner. 500 μl of 6M GITC-4% BME solution is added to each human brain sample. The sample is triturated vigorously through a syringe approximately 5 times to thoroughly disrupt the sample. If the sample is too large to pass through the syringe, an initial homogenization is performed using a hand-held Omni homogenizer. This is followed with using a syringe. 5.5 ml of TRIZOL is added to the sample. The sample is capped and thoroughly mixed with TRIZOL by quick inversion. The sample is placed on ice. The TRIZOL homogenates are transferred to pre-spun 15 ml PLG tubes. 2.4 ml chloroform is added and the tube is capped. The tube is shaken vigorously for 15 to 20 seconds and incubated on ice for 10 min. The samples are spun at 4300 rpm (4560×g) for 7 minutes at 4° C. The upper aqueous phase is transferred by pouring into a clean labeled 15 ml conical tube and place at on ice. The RNA is isolated using any standard RNA isolation procedure.

### Example VI

### Method for Predicting the Bone Marrow Toxicity of a Test Agent Using the Disclosed Methods for Isolating High-Quality RNA

The methods for isolating high quality RNA of the present invention can be used to test the bone marrow toxicity of a test agent. This is because the methods of the present invention do not require pre-lysis of red blood cells prior to lysis of the rest of the cells in the sample. Pre-lysis of RBCs prior to lysis of the rest of the cells in the sample results in the loss of gene expression information that is contained in immature or developing RBCs.

One way to predict the toxicity of a test agent using the present methods is to determine the gene expression profile of a sample of bone marrow progenitors that has been exposed to a test agent and compare this profile to a sample of bone marrow progenitors that has not been exposed to the test agent. Because the methods of the present invention do not require pre-lysis of red blood cells, the gene expression information contained in immature RBCs that have not shed their nuclei will be captured. That is to say, the high-quality RNA that is isolated using the methods of the invention will include RNA from immature and developing RBCs.

If no RBCs are present in the sample that has been exposed to the test agent, this will be an indication that the agent is toxic to bone marrow because the bone marrow does not produce RBCs or the bone marrow produces are reduced population RBCs.

Although the present invention has been described in detail with reference to examples above, it is understood that various modifications can be made without departing from the spirit of the invention. Accordingly, the invention is limited only by the following claims. All cited patents, patent applications and publications referred to in this application are hereby incorporated by reference in their entirety.

What is claimed is:

1. A method of isolating high quality RNA from a cell or tissue sample comprising:
a) suspending the cell or tissue sample in a buffer without pre-lysing red blood cells in the sample,
b) lysing the cell or tissue sample and
c) isolating high quality RNA from the lysed sample.

2. A method for isolating high quality RNA from a cell or tissue sample comprising:
a) suspending the cell or tissue sample in a buffer without pre-lysing red blood cells in the sample,
b) suspending cells in a solution of a chaotropic agent,
c) adding a cell lysis reagent and
d) isolating high quality RNA.

3. A method of claim 2 wherein said suspending in step a) is carried out using a pipette or a syringe attached to a needle.

4. A method of claim 2 wherein said chaotropic agent is a solution of about 4 Molar guanidine thiocyanate.

5. A method of claim 4 wherein said solution of guanidine thiocyanate contains about 1% 2-mercaptoethanol.

6. A method of claim 4, wherein said cell lysis reagent is TRIZOL.

7. A method of claim 2 wherein said cells are epithelial cells.

8. A method of claim 2 wherein said cells are fibroblasts.

9. A method of claim 2 wherein said cells are bone marrow cells.

10. A method of claim 2 wherein said cells are brain cells or brain tissue.

11. A method of claim 10 wherein said brain cells or brain tissue is human.

12. A method for isolating high quality RNA from bone marrow cells comprising the steps of:
a) suspending bone marrow cells in a buffer without lysing red blood cells in the sample,
b) combining bone marrow cells with an approximately 4 Molar solution of guanidine thiocyanate containing approximately 1% 2-mercaptoethanol,
c) suspending said bone marrow cells in said solution of step b) using a pipette or a syringe attached to a needle,
d) adding TRIZOL reagent and
e) isolating high quality RNA.

13. A method for isolating high quality RNA from bone marrow cells comprising the steps of:
a) suspending cells in a buffer by repeated pipetting or repeated syringing without lysing red blood cells present in the sample,
b) centrifuging the solution of step a) to form a pellet,
c) resuspending said pellet in a buffer by repeated pipetting or repeated syringing,
d) adding TRIZOL reagent and
e) isolating high quality RNA.

14. A method for isolating high quality RNA from brain cells or brain tissue comprising the steps of:
a) suspending brain cells or brain tissue in a buffer without lysing red blood cells in the sample,
b) suspending said brain cells or brain tissue in a solution of about 6M guanidine isothiocyanate containing about 4% beta-mercaptoethanol by repeated syringing or pipetting,
c) adding TRIZOL to the sample and
d) isolating high quality RNA.

15. A kit for isolating high quality RNA from a cell sample comprising
a) a vial or container of a solution of a chaotropic agent,
b) a vial or container of a solution of a cell lysis reagent,
c) syringes and syringe needles and
d) a package insert with instructions for isolating high quality RNA from a cell sample.

16. A kit of claim 15 wherein said chaotropic agent is a solution of about 4 Molar guanidine thiocyanate.

17. A kit of claim 15, wherein said solution of a chaotropic agent also contains about 1% by volume 2-mercaptoethanol.

18. A kit of claim 15 wherein said chaotropic agent is a solution of about 6 Molar guanidine thiocyanate.

19. A kit of claim 15 wherein said solution of a chaotropic agent also contains about 4% by volume 2-mercaptoethanol.

20. A method for predicting the bone marrow toxicity of an agent comprising:
a) obtaining a cell sample that has been previously treated with a test agent or isolated from an organism treated with a test agent,
b) isolating high quality RNA from the sample without pre-lysing red blood cells present in the sample prior to lysing the rest of the cells in the sample,
c) isolating high quality RNA from a cell or tissue sample that has not been exposed to the test agent, without pre-lysing red blood cells present in the sample and
d) correlating differences in the gene expression profiles between the two samples with the toxicity of the substance.

21. A method of claim 20 wherein said cell sample is bone marrow progenitors.
